(19) United States
(12) Patent Application Publication
(10) Pub. No.: US 2026/0308107 A1
(43) Pub. Date: Oct. 8, 2026

(54) METHOD OF SYNTHESIS

(71) Applicant: MITOCHOLINE LTD, London (GB)

(72) Inventor: Larisa Andreeva, London (GB)

(21) Appl. No.: 19/481,509

(22) PCT Filed: May 3, 2024
(86) PCT No.: PCT/GB2024/051170

§ 371 Date: Nov. 4, 2025

(30) Foreign Application Priority Data
May 5, 2023 (GB) 2306670.7
Nov. 16, 2023 (GB) 2317586.2

(51) Int. Cl. C07C 213/08; C07C 55/10; C07C 215/40

(52) U.S. Cl. CPC C07C 213/08; C07C 55/10; C07C 215/40

(57) ABSTRACT

The present invention provides methods of synthesis of di-choline succinate (DISU) and DISU formulations that are slightly hygroscopic or non-hygroscopic. The DISU formulations of the invention are suitable as food products or dietary supplements.

### FIELD OF THE INVENTION

The present invention relates to methods of synthesis of di-choline succinate and food products and food supplements comprising di-choline succinate obtainable by the methods.

### BACKGROUND OF THE INVENTION

Choline is an essential nutrient that is naturally present in some foods and available as a dietary supplement. Choline is a source of methyl groups needed for many steps in metabolism. The body needs choline to synthesize phosphatidylcholine and sphingomyelin, two major phospholipids vital for cell membranes. In addition, choline is needed to produce acetylcholine, an important neurotransmitter for memory, mood, muscle control, and other brain and nervous system functions. Choline also plays important roles in modulating gene expression, cell membrane signalling, lipid transport and metabolism, and early brain development.

Choline, in a form of a salt of succinic acid (di-choline succinate or DISU), has been shown to be a potent sensitizer of the neuronal insulin receptor (Storozhevykh T., et al (2008) BCM Neurocsi. doi: 10.1186/1471-2202-8-84). Further, compositions of di-choline succinate with nicotinamide are synergistically effective for increasing the levels of both NAD, ATP, and phosphocreatine in the brain cells (WO2019002858). Recently, it has been reported that DISU is also potent enhancer of skeletal muscle function (WO2022254185) and it has also been shown that DISU is the most potent in supporting one carbon metabolism in humans, compared to two other choline salts, choline bitartrate and choline chloride (WO2023026043).

Food supplementation with choline has been proven to be beneficial for health of both humans and domesticated mammals. Production of several salts of choline suitable as food supplements, like choline chloride, choline bitartrate or choline citrate/dihydrogen citrate, is well established and food supplements comprising these salts are commercially available.

Methods of preparation of crystalline DISU at lab scale have been described in WO2001076583 and in WO2009022933.

### SUMMARY OF THE INVENTION

A method of producing di-choline salt of succinic acid (DISU), comprising
obtaining a solution of choline in water or in a water miscible organic solvent selected from methanol, ethanol, 1- or 2-propanol, wherein the choline solution comprises one or more o-ethoxylated choline derivatives, wherein the amount of said one or more ethoxylated choline derivatives is above 0.3 wt. %
treating the choline solution with activated carbon (AC), wherein the weight ratio of AC to choline is 100-1:1-100;
adding succinic acid, or succinic acid salt to the choline solution, wherein the molar ratio of choline to succinate is between around 2:1, preferably between around 2:1.01 and around 2:1.1, and allowing succinate anion to react with choline cation for at least 10 min at ambient temperature, thereby producing DISU dissolved in water or in the water miscible organic solvent;
wherein the produced solution of DISU contains
at least 40 wt. % DISU, preferably 50 wt. % DISU, and
at most 0.3 wt. % of the one or more o-ethoxylated choline derivatives, preferably below 0.3 wt. %.

A second aspect of the invention relates to a liquid formulation comprising at least 40 wt. % DISU, preferably at least 50 wt. % DISU, and at most 0.3 wt. %, preferably less than 0.3 wt. %, of one or more o-ethoxylated choline derivatives.

A third aspect of the invention relates to a solid formulation comprising DISU and a hygroscopicity controlling agent (HCA), wherein the wt./wt. ratio of DISU:HCA is from 1:3 to 1:10, and wherein the formulation comprises at most 0.3 wt. %, preferably less than 0.3 wt. %, of one or more o-ethoxylated choline derivatives.

### DETAILED DESCRIPTION OF THE INVENTION

The present invention provides a simple robust industrially scalable and environmentally friendly method of synthesis of di-choline succinate (DISU) and preparing a non-hygroscopic DISU concentrated, wherein the method comprises reacting choline (base or salt) with succinate (acid or salt), preferably, in water and obtaining a DISU material that comprises at least 40 wt. % DISU and at most 0.3 wt. % of o-ethoxylated choline derivatives.

Synthesis of DISU has been described previously. WO2001076583 describes a method comprising a reaction of choline hydroxide (methanol solution) with succinic acid and crystallization of DISU from the reaction medium by using ter-butanol. Another method described previously (WO2009022933) comprises a sequence of steps to obtain crystalline DISU. Both methods employ crystallization from organic solvents as means to obtain DISU of purity that is suitable for pharmaceutical use. However, the final product, crystalline DISU, is not a stable formulation as it is highly hygroscopic and therefore the crystalline DISU material demands particular handling and storage conditions to avoid uncontrolled accumulation of water. Therefore, the described methods and DISU product obtained by these methods are not readily applicable for a large scale industrial manufacturing and long-term shelf storage of the compound, and, additionally, they have a drawback of being environmentally unfriendly due to use of large amount of organic solvents.

The present invention overcomes disadvantages of the above mentioned methods and provides a simple method of production of a food grade di-choline succinate (DISU), wherein the method comprises a step of reacting choline cation (“choline”) with divalent succinate anion (“succinate”) in water or a water miscible organic solvent selected from methanol, ethanol, 1-propanol or 2-propanol, wherein water is a preferred reaction medium, and thereby obtaining a concentrated DISU solution, wherein the content of DISU is at least 40 wt. %, preferably around 50 wt. %. or more. The DISU solution of the invention has purity comparable with a crystalline DISU as it comprises very low amounts of impurities such as trimethyl amine (TMA), ethylene oxide (EO), and derivatives thereof, in particular, it contains very low amounts of o-ethoxylated by-products of choline synthesis from TMA and EO. These o-ethoxylated by-products represent residual complexity (RC) of choline product of the reaction of TMA with EO. The term “residual complexity” (RC) process in the context of the invention means the subtle but significant convolution of major chemical species in materials that originate from biochemical or synthetic reaction mixtures (see go.uic.edu/residualcomplexity). The RC compounds comprised in choline hydroxide produced from TMA and EO are formed, first, via reaction of choline with EO and, then, via further reaction of the obtained choline-EO derivative with EO, like presented below:

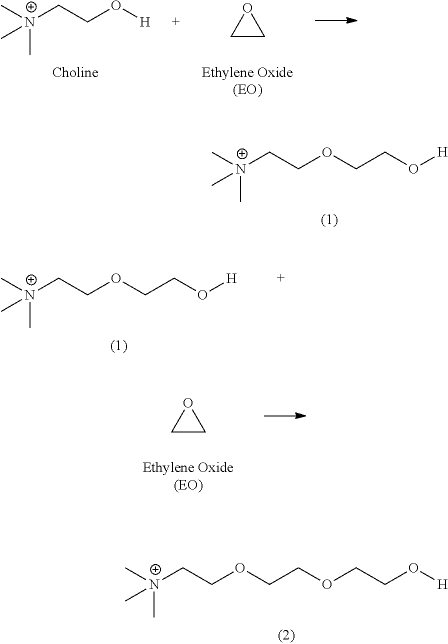

The reaction can continue and several structurally similar RC compounds can be produced. Accordingly, the final choline hydroxide product obtained following the reaction of TMA and EO may contain a pool of structurally related o-ethoxylated derivatives of choline (RC compounds) comprising one or more compounds shown below:

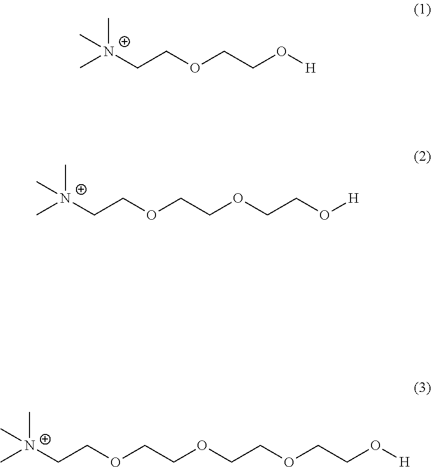

Typically, these by-products are present in the choline hydroxide solution in the amounts significantly higher than 0.3 wt. %, often, typically from around 3 wt. % to around 10-15 wt. %. These by-products are undesirable in food products and therefore they have to be removed from either initial choline solution used in the method of the present invention for the synthesis of DISU, or from final DISU products.

The methods for reducing the amount of these by-products in choline hydroxide obtained by the above described reaction have been described in the prior art. Most of these methods employ either lower reaction temperatures in the reaction between TMA and EO and/or reduced amount of EO relative to the amount of TMA used for the synthesis (WO2013098575). However, the efficiency of this method is not sufficient and the obtained choline hydroxide needs further processing to remove these by-products, in case a food grade quality of choline products are desirable. Another method that has also been previously described is to prepare choline hydroxide from choline chloride, where choline chloride is purified by use of a weakly acidic cationic resin (U.S. Pat. No. 3,522,296). The process seems to be more efficient, however, it is rather complex and laborious. Still, another method to obtain a high purity choline hydroxide is to prepare it from other choline salts which are obtained by crystallization. However, this method is too complex and, on the top, it is too expensive to be considered as suitable industrial manufacturing of food grade choline salts, like DISU, and other choline derivatives.

This invention provides an alternative method that allows to produce DISU, that comprises at most around 0.3 wt. %, preferably less than 0.3 wt. %, such as around 0.05-0.1 wt. %, preferably less, e.g. 0.015-0.035 wt. % of the RC compounds of choline hydroxide synthesis (i.e. o-ethoxylated choline derivatives like one or more compounds (1), (2) and (3)), and, also, very little amounts, if any, of TMA and EO, or derivatives thereof, like dioxane or ethylene glycol. Surprisingly, the inventors found that treatment of a choline solution comprising the above mentioned o-ethoxylated choline derivatives (RC compounds) with activated charcoal, where the weight ratio of activated charcoal/carbon to choline is 100-1:1-100, is very effective to remove these compounds. Preferably, at least 1 wt. part of active charcoal (AC) is be used to treat 10 wt. parts of choline (i.e. 1:10 wt/wt AC to choline, i.e. 1 g AC for the treatment of 10 g of choline), e.g. 1:5 wt/wt or 1:3 wt/wt. AC to choline. However, the relative amount of AC used for the treatment may vary in different embodiments. In different embodiments a preferred wt./wt. ratio of AC to choline could be in the range of 1-50:1 wt./wt.; in other preferred embodiments the ratio could be in the range of 50-100:1 wt./wt.; in other preferred embodiments, the ratio of AC to choline may be 1:1-50 or 1:50-100 wt./wt. The amount of AC to be used to efficiently remove the above described impurities from the choline material would depend on the amount of these compounds (o-ethoxylated derivatives of choline) present in the choline material. The amount of RC compounds in the choline solution can be determined by any suitable method of the prior art, e.g. one that is described in Achanta, P. C., et al. (2022) J Pharm Biomed Anal., 214:114709; doi: 10.1016/j.jpba.2022.114709. It is understood that all values within the indicated ranges are included in the scope of the invention. The AC (also termed herein as “activated carbon”) treatment significantly reduces the amount of o-ethoxylated choline by-products (also termed herein as “o-ethoxylated impurities”), such as by more than 10 fold, such as by 100 folds or more, such as by 300-1000 folds or more, and provides a choline hydroxide comprising at most 0.3 wt. % of these by-products. This simple, but surprising approach solves the problem of reducing the amount of undesirable RC products, such as o-ethoxylated choline derivatives, comprised in the starting choline material in DISU produced from this choline material without application of any other steps typically used to obtain DISU of high purity, like crystallization. Still, the method allows to crystallize DISU from the produced DISU material, if it is desirable, for other purposes, e.g. to obtain solid non-hygroscopic formulations of DISU described herein.

The method described herein can also be advantageously used for the production of other salts of choline that are widely used as food supplements, e.g., choline chloride, choline bitartrate, choline citrate/dihydrogen citrate etc.

The aspects and embodiments of the present invention are described below and illustrated by non-limiting working examples. All terms and definitions presented through the text of the specification relate to all aspects and embodiments of the invention, unless specified otherwise. It is understood that a value defined by a range of values includes all and each single value of the range.

The term “about”/“around” means a deviation from the indicated value by 0.01% to 10%, such as from 0.5% to 5%.

The terms “less” and “below” used in the present context as synonyms.

The term “at most” means in the present context as all values that are less than the indicated.

The term “choline” (“choline cation”) means a compound having the chemical formula C₅H₁₄NO⁺ (CAS No. 62-49-7). In different embodiments of the invention, choline could be in a form of base, i.e. choline hydroxide, or in a salt form, e.g. choline chloride, choline (bi)carbonate, etc.

The term “succinate” in the present context means the divalent anion of succinic acid (CAS No. 110-15-6).

The terms “di-choline succinate”, “choline succinate salt (2:1)” and “DISU” are interchangeable and mean the molecule of formula (I) (CAS No. 109438-15-5):

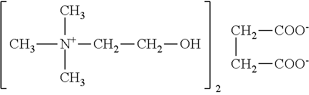

The term “DISU material” means a composition comprising a certain amount of DISU compound as above, e.g. a solution of DISU in aqueous or water miscible solvent medium, wet crystalline DISU, a composition comprising DISU and an hygroscopicity controlling additive (HCA). The term “humidity hygroscopicity additive/agent” means a hygroscopic material that serves to maintain a state of dryness. In the content of the present invention the HCA compounds are desiccants.

The term “molar ratio” means relative amounts of molecules in a compound, e.g. in di-choline succinate the molar ratio of choline to succinate is 2:1.

The term “water miscible organic solvent” means that the solvent can be mixed with water at any ratio without separation of two phases. The term “aqueous solution” or “water solution” means water comprising one or more dissolved molecules, e.g. choline hydroxide.

The term “activated carbon (AC) (interchangeably termed herein as “activated charcoal”) means a carbonaceous adsorbents synthesized from a carbonaceous material with high carbon content, low ash content, and significant volatiles matter through physical, chemical or a combination of both treatment.

The term “v/v” means volume to volume and is in the present context used to describe a ratio between two liquids. The term “wt. %” is interchangeable with “wt/wt” and means mass/weight fraction of a compound of interest in the composition.

The term “derivative” means, a compound that is derived from a similar compound by a chemical reaction.

The term “by-product” means a secondary product derived from a production process, manufacturing process or chemical reaction.

The term “impurity” means a chemical substance inside a confined amount of liquid, gas, or solid. They differ from the chemical composition of the material or compound.

The term “degradation product” means chemicals resulting from partial decomposition or chemical breakdown of substances.

The present invention preferably relates to a food grade purity of DISU. In context of the present invention, the term “food grade purity” means that DISU obtained by the process described herein contains impurities/by-products/degradation products, such as trimethylamine (TMA), ethylene oxide (EO), ethylene glycol, glycol, chloroethanol, dioxane, in the amount of around 500 ppm or less (i.e. 0.05 wt. % for each impurity), preferably, between about 200-500 ppm or less, such as between around 50-200 ppm, between around 10-50 ppm, or less, such as around 1-5 ppm, or less. The term “ppm” or “parts per million” means a unit that is typically used to described very small concentrations of a substance, e.g. in a mixture of different substances. 1 ppm is equal to 1 milli of other units such as a milligram per liter of solution, milligram per kilogram, etc. The later impurities are typical for choline synthesized from TMA and ethylene oxide and often present in the compound. According to invention, these impurities are to be removed (partially or completely) either from choline as initial material for DISU synthesis before the synthesis or from DISU obtained from choline containing these impurities after the synthesis. In a preferred embodiment, the impurities are removed (fully or partially) from choline intended as the initial material for the DISU synthesis. Some impurities, e.g. TMA or EO, can be removed from a choline aqueous solution e.g. by distilling the solution in vacuo with a nitrogen sweep at a pressure of 20 mm of Hg and a pot temperature of ~38° C. Water can be added to the still pot as needed to maintain the initial volume. Distillation is continued until the TMA level in the pot liquid is reduced below 15 ppm, preferably below 10 ppm, such as around 5 ppm. Alternatively, the impurities, especially non-charged or negatively charged molecules may be removed by treating the choline solution with an ion exchanger, e.g. cation exchanger, preferably a weak acidic cation exchanger, e.g. as described in U.S. Pat. No. 3,522,396. Passage over a weakly acidic cationic resin has the advantage of purifying the choline by eliminating the aminated impurities comprising volatile amines, such as mono-, di- and trimethylamine and fixed amines such as the amines formed by o-ethoxylation of the first two mentioned. All these amines are rather hydrophobic and weak bases in comparison with choline. By passing an excess of solution over the cationic resin, the amines originally fixed are gradually displaced by the choline, for which the resin has a greater affinity, and they pass into the effluent. Additionally, non-charged impurities are also to be removed in this passage. Further, the invention provides a method for removal other organic impurities from choline produced via reaction of TMA and EO, such as o-ethoxylated choline by-products (RC compounds), wherein the choline solution is be treated with AC. AC can efficiently absorb these impurities and significantly reduce their amount in the choline material intended for the synthesis of DISU to the desired amount of at most 0.3 wt. %. The AC treatment can be done before or following the treatment with weak cationic resin and/or distillation. In a preferred embodiment o-ethoxylated compounds are removed by treating a choline hydroxide solution with AC following the step of removal of TMA and EO by distillation as described above in one or repeated treatment, as described further in specification.

The term “aqueous medium” means water or water solution of one or more chemical substances.

The term “water miscible solvent” in the context of the invention means an organic solvent that can be mixed with water and form homogeneous mixture (i.e. without substantial separation of phases), such as, in one embodiment of the invention, an alcohol comprising 1 to 3 carbon atoms, such as methanol, ethanol, propanol (including 2-propanol and 1-propanol), or, in another embodiment of the invention, acetone. A first aspect of invention relates to a method of producing a di-choline salt of succinic acid (DISU), comprising
obtaining a solution of choline in water or in a water miscible organic solvent selected from methanol, ethanol, 1- or 2-propanol, wherein the choline solution comprises one or more o-ethoxylated choline derivatives, wherein the amount of said one or more ethoxylated choline derivatives is above 0.3 wt. %;
treating the choline solution with activated carbon (AC), wherein the weight ratio of AC to choline is 100-1:1-100, preferably wt./wt. ratio of AC:choline is 50-1:1-50, such as 10-1:1-1-10;
adding succinic acid, or succinic acid salt to the choline solution, wherein the molar ratio of choline to succinate is between around 2:1, such as between around 2:1.01 and around 2:1.1, and allowing succinate anion to react with choline cation for at least 10 min at ambient temperature, thereby producing DISU dissolved in water or in the water miscible organic solvent;
   wherein the produced solution of DISU contains
at least 40 wt. %, preferably around 50 wt. % DISU, and at most 0.3 wt. %, preferably below 0.3 wt. %, of one or more of said o-ethoxylated derivatives of choline, e.g. around 0.2 wt. %, such as between 0.05 wt. % and 0.15 wt. % or less, such as between around 0.015-0.035 wt. %.

According to the invention, reducing the amount of one or more o-ethoxylated derivatives of choline, especially, mono-, di- and tri-ethoxylated choline, comprises treating a choline solution, preferably, the solution that have been processed through distillation to remove residual TMA and EO, with activated carbon (AC). In different embodiments AC could be used either in a powdered form, granulated or packed in a cartridge. AC is commercially available from multiple manufacturers. Preferably, AC is used in a packed-in-cartridge form. The amount of AC used for the treatment is at least 1 wt. part for 10 wt. parts of choline (i.e. 1:10 wt/wt, like 1 g AC for treatment of 10 g of choline), such as 1:5 wt/wt or 1:3 wt/wt. In some embodiments, the AC treatment of choline solution may be repeated as many times as needed to obtain the final choline solution where the amount of these by-products is at most 0.3 wt. %, preferably less than 0.3 wt. %, such as less than 0.2 wt. %, e.g. between around 0.05-0.15 wt %, such as between around 0.015-0.035 wt. %. According to the invention, the AC treatment reduces the amount of these impurities in the final choline solution (i.e. the solution that is used for DISU synthesis) by at least 10-100 fold, like by 100-500 folds, or 500-1000 folds.

Preferably, before treatment with AC, the choline solution is distilled in vacuo with a nitrogen sweep at a pressure of 20 mm of Hg and a pot temperature of ~38° C. to remove TMA and EO.

The choline solution in one preferred embodiment is a solution of choline hydroxide in water. However, embodiments where choline hydroxide is dissolved in a water miscible solvent are also included in the scope of the invention. Preferable water miscible solvents of the invention include methanol, ethanol, and 1- or 2-propanol, or mixtures thereof. In embodiments, when choline hydroxide is dissolved in a water miscible solvent, the DISU solution obtained in the synthesis is a solution of DISU in the corresponding water miscible solvent, wherein the content of DISU is at least 50 wt. %. These embodiments of DISU solution may have advantage when a solid non-hygroscopic formulations of DISU is to be prepared. The organic solvent based DISU solutions may facilitate dissolving hygroscopicity controlling additives (HCA) in these solutions and drying the prepared blend to obtain solid formulations of DISU, such as the one described in the specification below.

Embodiments of the invention also include use of a choline solution like a solution of choline salt in water, or in a water miscible solvent selected from methanol, ethanol or 1- or 2-propanol, or mixtures thereof. In these embodiments, the method of the invention comprises.
adding a base to the choline salt solution, thereby obtaining a solution comprising choline hydroxide and a salt,
adding succinic acid to the solution of choline hydroxide and the salt, wherein the molar ratio of choline cation to succinate anion is around 2:1, such as between around 2:1.01 and around 2:1.1, and allowing succinate anion to react with choline cation for at least 10 min at ambient temperature to form DISU;
removing the salt from the DISU solution.

A preferable choline salt of the invention is choline bicarbonate or choline chloride or choline carbonate. These salts of choline are commercially available. A base to be used for converting the choline salt to choline hydroxide is preferably an inorganic base, such as sodium hydroxide, potassium or ammonium hydroxide. In preferred embodiments, the salt obtained in this conversion is either sodium or potassium carbonate or chloride, with the proviso that the choline solution is choline bicarbonate/choline carbonate or choline chloride, respectively, and the base is sodium or potassium hydroxide; and the salt is ammonium carbonate, with the proviso that choline solution is choline bicarbonate/choline carbonate and the base is ammonium hydroxide. The obtained salt is an undesirable by-product of the described reaction and is to be removed from the choline hydroxide solution before or after reaction with succinic acid. Salts like sodium or potassium carbonate or chloride are not soluble in organic solvents, therefore it could be advantageous to add an organic solvent to the reaction mixture of choline hydroxide and succinic acid to precipitate the salt. Accordingly, in one preferred embodiment, succinic acid added to the solution of choline hydroxide as a solution of succinic acid in ethanol, methanol or 1-, or 2-propanol. In another preferred embodiment, the choline solution is a solution of choline salt, like choline chloride or choline (bi)carbonate, in methanol, ethanol, or 1- or 2-propanol, and succinic acid added is a water solution of succinic acid or crystalline succinic acid. The salt precipitated could be easily removed from reaction medium by filtration. In one preferred embodiment, the choline salt is choline (bi)carbonate solution in water and the base is ammonium hydroxide. The obtained by-product salt, ammonium carbonate, in this embodiment, may be removed by heating the solution containing thereof up to around 55° C. In one embodiment, ammonium carbonate is removed from choline hydroxide solution before adding succinic acid, in another embodiment, ammonium carbonate is removed from the reaction mixture containing DISU.

According to the invention, the obtained DISU material comprises at least 40 wt. % DISU, preferably around 50 wt. % DISU. The DISU material could be concentrated, e.g. by vacuum evaporation, to up to around 70 wt. % DISU, such as around 69 wt. % DISU (corresponds to DISU octahydrate, i.e. DISU with 8 molecules of water) or around 72 wt. % DISU (corresponds to DISU heptahydrate, i.e. DISU with 7 molecules of water). This concentrated DISU material corresponds to octahydrate DISU which is a very stable formulation, i.e. it has at least 6 month shelf life without significant change in the composition in conditions of high humidity and ambient temperature of 25-40° C. The produced 40-70 wt. % solution of DISU according to the invention is slightly hygroscopic i.e. the weight gain of the DISU material maintained for 24 hours at 25° C.±1° C. and 80%±2% relative humidity (RH) is at most 2% (wt/wt), preferably, around 1.5%, such as around 1% or less, e.g. around 0.2-0.5% (wt/wt).

In one embodiment, reducing content of liquid in the obtained DISU material could be done by precipitation of DISU. Preferably, the DISU precipitate is obtained by
adding at least 2 volumes of acetone to the DISU solution and incubating the mixture for 3-6 hours at 10-15° C.;
collecting the DISU precipitate by using the Nutsche filtration;
drying the DISU precipitate collected in the Nutsche filter.

According to the invention, the dried DISU material is preferably used for preparation of non-hygroscopic dry formulations of DISU. This preparation would typically comprise
dissolving the dried DISU in an organic solvent, preferably methanol, ethanol, 1-propanol, 2-propanol, or a mixture thereof;
mixing the DISU solution with one or more hygroscopicity controlling agents (HCA),
removing the solvent by drying the mixture, and thereby obtaining a non-hygroscopic solid formulation of DISU.

According to the invention, the one or more HCA is/are preferably selected from microcrystalline cellulose, ethyl cellulose, hydroxypropyl cellulose, hydroxypropyl methyl cellulose, ethyl methyl cellulose, or a mixture thereof.

Typically, DISU is mixed with an HCA in the wt./wt. DISU:HCA ratio from 1:3 to 1:8 preferably between around 1:3-1:4 wt./wt. DISU:HCA ratio.

Accordingly, one aspect of the invention is a slightly hygroscopic or non-hygroscopic formulation of DISU material obtained by the method of the invention. In one preferred embodiment, the slightly hygroscopic formulation of DISU is around 30 wt. % water or water miscible organic solvent selected from methanol, ethanol or 1- or 2-propanol. In one preferred embodiment, a solid non-hygroscopic formulation of DISU, wherein the formulation comprises DISU and HCA in the wt./wt. ratio from about 1:3 to about 1:8, preferably between about 1:3 to 1:4 wt./wt. DISU to HCA. The HCA is preferably one or more compounds selected from microcrystalline cellulose, ethyl cellulose, hydroxypropyl, or mixtures thereof.

Different additional embodiments of the above method and compositions of the invention are further described below.

Synthesis of DISU is typically conducted in a medium comprising around 2 molar parts of choline (i.e. choline cation) and around 1 molar part of succinate (i.e. di-valent succinate anion), i.e. the molar ratio of choline to succinate in the reaction mixture is around 1.9-2:1-1.1, such as 2:1, 1.95:1, 2:1.01, etc. Preferably, succinate (divalent anion of succinic acid) is present in the reaction medium in a some molar amount excess with regard to the molar amount of choline cation in order to secure formation of di-choline succinate and avoid formation of mono-choline succinate.

The reaction between succinate and choline is typically takes at least 10 min at ambient temperature, however, reaction time may be extended to longer period of times, if this extension required for convenience of the manufacturing. The extension of reaction time does not lead to formation of undesirable by-products.

The initial compounds of the reaction are a solution of choline hydroxide or a solution of a choline salt and succinic acid, and the reaction is preferably performed in an aqueous medium. In another preferred embodiment, succinic acid is preferably used in its crystalline form and choline hydroxide/salt as an alcohol solution, such as methanol, ethanol or 2-propanol solution. Both crystalline succinic acid and aqueous and alcohol solutions of choline are commercially available. Preferably, the commercial compounds to be used for the reaction are of food grade purity. Alternatively, choline hydroxide solution could be purified before the reaction by using the methods described above.

Accordingly, in one embodiment, the method of synthesis of DISU according to the invention comprises
Providing a choline hydroxide aqueous solution and crystalline succinic acid,
Optionally, purifying the a solution of choline hydroxide to food grade quality;
1 Adding succinic acid to the solution of choline hydroxide, so that the mixture of the choline hydroxide and succinic acid comprises around 2 molar parts of choline and around 1 molar part of succinate, preferably 1.01-1.1 molar parts of succinate per 1 molar part of choline, thereby reacting choline with succinate and obtaining a solution of DISU in water.

In another embodiment, the initial choline hydroxide solution is in a methanol, ethanol or 1- or 2-propanol, and the method of synthesis of DISU according to the invention comprises
providing a choline hydroxide solution in methanol, ethanol or 1-propanol or 2-propanol, or a mixture thereof, and crystalline succinic acid,
Optionally, purifying the a solution of choline hydroxide to food grade quality as described above;
1 Adding succinic acid to the solution of choline hydroxide, so that the mixture of the choline hydroxide and succinic acid comprises around 2 molar parts of choline and around 1 molar part of succinate, preferably 1.01-1.1 molar parts of succinate per 1 molar part of choline, thereby reacting choline with succinate and obtaining a solution of DISU in methanol, ethanol, 1-propanol or 2-propanol, or a mixture thereof.

In one embodiment, the method of synthesis of DISU according to the invention comprises providing a solution of choline hydroxide that comprises below 0.3 wt. % of o-ethoxylated derivatives of choline that is further used in the process of synthesis of DISU. Such solution of choline hydroxide is provided according to the invention by treating a solution of choline hydroxide that comprises above 0.3 wt. % of o-ethoxylated derivatives of choline with AC, as described above; or, alternatively, by reacting an alcohol solution, e.g., 2-propanol solution, or aqueous solution of food grade choline chloride with sodium hydroxide. Accordingly, in this embodiment the method of the invention comprises
Providing a solution of choline chloride in methanol, ethanol, 1- or 2-propanol,
Adding sodium hydroxide to the solution of choline chloride, thereby obtaining a solution of choline hydroxide in 2-propanol and precipitate of sodium chloride;
1 Removing sodium chloride precipitate from the solution of choline hydroxide, and
Adding succinic acid to the solution of choline hydroxide, so that the mixture of the choline hydroxide and succinic acid comprises around 2 molar parts of choline and around 1 molar part of succinate, thereby reacting choline with succinate and obtaining a solution of DISU in 2-propanol.

Choline chloride of food grade quality is commercially available and typically does not need additional purification. However, if necessary, choline chloride may be purified using a weak acidic cation exchanger and treated with AC (as described above).

Choline chloride dissolved in water or in an alcohol, preferably 2-propanol, is converted in choline hydroxide by adding an equimolar amount of sodium hydroxide, thereby the solution of choline hydroxide in water or alcohol is provided.

The reaction between choline chloride and sodium hydroxide may be performed at around room temperature. However, in some embodiments, the reaction can also be performed at 25-50° C. The reaction time would typically be around 3 to 6 hours.

Sodium chloride precipitate formed in reaction between choline chloride and sodium hydroxide in 2-propanol medium or aqueous medium is removed from the reaction medium containing choline hydroxide before this solution of choline hydroxide proceeds to the further step—the step of reacting choline with succinic acid.

To remove sodium chloride precipitate formed in 2-propanol reaction media, the reaction media comprising dissolved choline hydroxide and a suspension of sodium chloride precipitate may be subjected to filtration, centrifugation, or another suitable procedure for separating precipitated material from liquid medium. In a preferred embodiment, the solution is filtered using Nutsche filtration. Following the filtration, the passed through filter liquid phase containing choline hydroxide is collected and proceeded to the step of synthesis of DISU in the reaction of choline with succinate. The precipitated sodium chloride material may be additionally washed with 2-propanol to extract the residual choline hydroxide, which is then to be combined with the major fraction collected following the filtration.

To remove sodium chloride from the aqueous reaction media comprising choline hydroxide and sodium chloride, both dissolved, an excess of a solvent, in which sodium chloride is insoluble and choline hydroxide is soluble, is added to the reaction medium. Preferably, the solvent is 2-propanol. Preferably, the aqueous reaction medium is treated to reduce the content of water, e.g., by airdrying, vacuum drying, water evaporation, nanofiltration, reverse osmosis, spray-drying, freeze-drying of the DISU solution, or other suitable for the purpose method, before addition of 2-propanol. Typically, around 7-10 volumes of 2-propanol are added to 1 volume of aqueous solution to precipitate the dissolved sodium chloride. Besides 2-propanol desalting of the aqueous reaction media, other methods of desalting known in the art could be applied.

On the next step of the process, succinic acid is added to the filtrated solution of choline hydroxide. Succinic acid used for the DISU synthesis may be a crystalline material or a succinic acid solution, preferably in 2-propanol. Typically, succinic acid in a solid form is added to the solution in the amount to achieve the molar ratio of choline to succinate of about 2:1.

Typically, in all the above embodiments of the method of invention, the reaction of choline and succinate is carried out at a room temperature (i.e. at around 19-25° C.) during from around 10-60 min to 2-3 hours at constant stirring. However, the reaction can be performed at another temperature as well, both below the indicated and above of the indicated could be applied (e.g. any temperature in the range of 10-50° C. could be used). The yield of DISU obtained in the reaction is typically around 100%.

In one preferred embodiment, both above methods may further comprise a step of reducing the content of the reaction media (i.e. the DISU solvent) in the obtained DISU material. The reduction in one embodiment may be done by obtaining a solid DISU, such as crystalline or amorphous solid DISU, or by obtaining a concentrated solution of DISU. Preferably, a concentrated solution of DISU is a slightly hygroscopic aqueous solution of DISU.

By the term “slightly-hygroscopic solution” is meant a solution that does not absorb significant amounts of water from surrounding environment, where “significant amounts” means an amount of absorbed water that increases the mass of the sample of the compound no more than by 2% following 24 hours storage of the sample at around 80% relative humidity and around 25° C. The term “concentrated solution” in the present context means an aqueous solution that contains more than 40 wt. % DISU, preferably more than 50 wt. % DISU, preferably, between 55-68 wt. % DISU, such as around 70 wt. % DISU, e.g. 69 wt. % DISU or 72 wt. % DISU. The concentrated DISU solution according to the invention is a slightly hygroscopic solution.

The procedure of obtaining of an aqueous slightly hygroscopic DISU solution, i.e. a solution containing more than 40 wt. % DISU, preferably at least 50 wt. %, such as between 55-68 wt. % DISU, such as around 70 wt. % DISU, e.g. 69 wt. % DISU or 72 wt. % DISU, may include one or more steps of reduction of water, e.g. by using airdrying, vacuum drying, water evaporation, nanofiltration, reverse osmosis, spray-drying, freeze-drying of the DISU solution, or other suitable for the purpose method. In one embodiment, a slightly hygroscopic aqueous DISU concentrated solution may be obtained from crystalline DISU. Crystalline DISU can be obtained from either an aqueous or alcohol solution of DISU, preferably from an alcohol DISU solution, preferably a DISU alcohol solution comprising 2-propanol, by mixing said DISU solution with acetone.

DISU synthesized by any of the above embodiments of the method may optionally be precipitated from the reaction medium in a crystalline form by mixing the reaction medium with acetone. Preferably, the reaction medium containing dissolved DISU is concentrated (5-20 folds) before mixing with the precipitating agent, preferably acetone. Removal of the excess of solvent can be done by any suitable method, e.g. evaporation. A 5-20 fold concentrated solution of DISU is then mixed with the equal volume of acetone, preferably with around 2 to 10 volumes of acetone, and maintained stirred at a temperature of around 10-15° C. for 2-3 hours. Preferably, DISU is precipitated from an alcoholic solution, such as 2-propanol or a mixture of 2-propanol with methanol, ethanol or 1-propanol.

Mixing a DISU solution with chilled acetone results in obtaining a crystalline DISU suspended in the reaction mixture. The suspension of crystalline DISU is first subjected to filtration to collect the solid. Preferably, the solid material is collected by using Nutsche filtration. The term “Nutsche filtration” means a batch filtration technique that uses vacuum and/or pressure in a closed vessel (“Nutsche filter”). The pressure/vacuum is used in combination with a filter plate fitted with the appropriate filter media to force liquid through media, forming a solid bed often referred as “cake”.

The DISU cake is then dried to remove the liquid. The dried DISU cake can be optionally repeatedly washed with chilled acetone and dried again.

Crystalline DISU is highly hydroscopic and needs to be kept under non-humid conditions. Dried DISU material collected from Nutsche filter should be immediately sealed in a tight container. If there is a need of a long term storage of DISU, crystalline DISU is preferably to be formulated as hepta- or octahydrate DISU (i.e. formulation comprising one DISU molecule and 7 or 8 molecules of water) which corresponds to a liquid composition of DISU containing around 70 wt. % DISU and 30 wt. % water. The concentrate can be prepared by adding an amount of water to the dried crystalline DISU to make a solution with the desired wt. % DISU directly in the Nutsche filter.

Alternatively, according to the invention, obtained crystalline DISU can be mixed with a compound which could control hygroscopicity of DISU in a solid mixed form. For example, dry crystalline DISU can be dissolved in an alcohol, preferably absolute ethanol, and mixed with a powder or an alcohol solution of microcrystalline cellulose, ethyl cellulose, hydroxypropyl cellulose, hydroxypropyl methyl cellulose, ethyl methyl cellulose, or a mixture thereof, and dried. The obtained dry formulation has much low hygroscopicity and therefore could be transported and stored without special precautions regarding ambient humidity. In one preferred embodiment, crystalline DISU is formulated in a mixture with microcrystalline cellulose and hydroxypropyl cellulose (or hydroxypropyl methylcellulose) in the w/w ratio 1:3-6:0.3-0.4 (DISU:microcrystalline cellulose:hydroxypropyl cellulose (or hydroxypropyl methylcellulose), correspondingly), The dry powder comprising a mixture of DISU and cellulose derivatives, may optionally further comprise an amount of succinic acid, which would stabilize DISU in the formulation, and silica, which would confer further control hygroscopicity of the composition. Typically, the amount of succinic acid added to the composition is about 2,5% of the DISU amount and the amount of added silica is about 7-10% of the DISU amount.

The solid dry formulations of DISU of the invention (as described above) are practically non-hygroscopic, i.e. the dry material stored 24 h at 80% relative humidity and 25° C. has not more than 0-0.012% w/w increase in mass. All components of these compositions are non-toxic and approved as food additives by EFSA and FDA therefore the compositions can be easily sealed in eatable capsules (e.g. gelatine or hydroxypropyl methyl cellulose (HPMC)) capsules and marketed as DISU dietary supplements. Such encapsulation of the described solid formulations of DISU further decreases the risk of humidity absorption by DISU without deteriorating its bioavailability. Non-limiting examples of such solid formulations of DISU are described in Examples.

Solutions and reaction media comprising choline hydroxide, choline chloride or other salts of choline, succinic acid and DISU may be treated with AC as many times as necessary to obtain desirable DISU material that contains at most 0.3 wt. % o-ethoxylated derivatives of choline.

### EXAMPLES

The working examples below are for illustration of the invention and do not limit the scope of the invention any way.

### Example 1. Manufacturing Process for DISU from Choline Chloride and Succinic Acid in Reaction Medium Comprising 2-Propanol

2-propanol was charged in a jacketed reactor equipped with temperature control system and agitator. The temperature was set to 25 to 50° C., and choline chloride (food grade) was added when the temperature reached 40±1° C. The mixture was kept under stirring until complete dissolution of choline chloride. Sodium hydroxide was then added, and the mixture was kept under stirring for 6 hours at a temperature of 25 to 50° C. Upon completion of the holding time, the reaction mass was filtered through a mono-plate filter. The sodium chloride sediment was pressed and rinsed with 2-propanol. The filtrate was transferred to a jacketed reactor equipped with temperature control system and agitator.

The filtrate from the previous step was heated at 25 to 50° C. Crystalline succinic acid was added (molar ratio around 1:2 succinate:choline) and completely dissolved and the mixture was kept under stirring for 3 hours. Upon completion of the holding time, the solution was filtered through an activated carbon filter cartridge.

The filtered solution was concentrated (by evaporation in the vacuum) to residual volume and transferred to a reactor containing around 3 vol acetone (pre-cooled at 10 to 15° C.). The distilled 2-propanol was transferred to a collecting vessel. The reaction mass was kept under stirring for 3 hours at a temperature of 10 to 15° C.

The formed sediment was filtered through a Nutsche filter, pressed, and rinsed with a 2-propanol:acetone (1:2) mixture. A moist sediment with residual solvent was obtained. Both the mother-liquors and the rinse solution were collected in a collecting vessel. Crude DISU was formed.

2-Propanol was added to the Nutsche filter containing the moist sediment of DISU to dissolve it. The solution of DISU in 2-propanol was transferred to a reactor containing acetone pre-cooled at around 10° C. The reaction mass is kept under stirring for 3 hours, maintaining the temperature within the range 10 to 15° C. The formed sediment was filtered through a Nutsche filter, pressed, and rinsed with a 2-propanol/acetone mixture 1:2). A moist sediment of DISU with residual solvent is obtained. Both the mother-liquors and the rinse solution were collected in a collecting vessel. The filtration cake was then dried under vacuum and nitrogen atmosphere. Purified DISU is formed.

After the drying step is complete, the dried DISU sediment on the Nutsche filter is either dissolved in purified water to the target DISU content value (at least 50 wt. %), or formulated as described in Example 4. The sample was analyzed by HPLC and Q-NMR and no o-ethoxylated impurities of choline were detected.

### Example 2. Manufacturing Process for DISU from Choline Hydroxide and Succinic Acid in Aqueous Medium

Aqueous solution of non-food grade choline hydroxide (45-55 wt. % choline) was distilled in vacuo with a nitrogen sweep at a pressure of 20 mm of Hg and a pot temperature of ~38° C. Following distillation, the solution was treated with powdered activated charcoal (AC) (10% wt/wt, 1 h stirring at ambient temperature), filtered and the filtrate was heated at 20-50° C. Crystalline succinic acid was added (molar ratio around 1:2 succinate:choline), and the mixture was kept under stirring for 15 min until all succinic acid was dissolved. Upon completion of the holding time, the solution was filtered through an AC filter cartridge, and a sample of the filtered solution was taken for analysis by HPLC and Q-NMR. The analysis showed that the sample contains around 50 wt. % DISU and around 0.1 wt. % o-ethoxylated derivatives of choline, TMA was not detected. The amount of the o-ethoxylated derivatives of choline in DISU sample was reduced by approximately 100 fold compared to their amount in the initial aqueous solution of choline hydroxide, and TMA was completely removed by the AC treatment.

The prepared 50 wt. % DISU solution was either kept at room temperature in a closed container, or used for preparation of DISU octahydrate material (60-70 wt. % DISU solution) or a solid non-hygroscopic formulation of DISU (see Example 4) as described below.

The filtered 50 wt. % DISU solution was then concentrated (by evaporation in the vacuum) to residual volume, mixed with 2-propanol (1 v/v) and transferred to a reactor containing acetone (3-5 v/v, pre-cooled at 10-15° C.). The reaction mass was kept under stirring for 3 hours at a temperature around 10° C.

The formed sediment was filtered through a Nutsche filter, pressed, and rinsed with a 2-propanol:acetone mixture (1:2 v/v). A moist sediment of DISU containing residual solvent was obtained. Both the mother-liquors and the rinse solution were collected in a collecting vessel.

2-propanol was added to the Nutsche filter containing the moist sediment of the crude DISU and DISU was thereby suspended. The DISU solution suspension in 2-propanol was transferred to a reactor containing acetone pre-cooled at 10 to 15° C. (1:2 v/v). The reaction mass is kept under stirring for 3 hours, maintaining the temperature within the range 10 to 15° C. The formed sediment was filtered through a Nutsche filter, pressed, and rinsed with a 2-propanol/acetone mixture. The moist sediment of DISU containing residual solvent was then dried. After the drying step was complete, the dried DISU sediment on the Nutsche filter was either dissolved in purified water to the target DISU content value (to at least 50 wt. %), or formulated as described in Example 4. The analysis of the DISU sample showed a further decrease in the amount of o-ethoxylated derivatives of choline to approximately 0.03-0.05 wt. %.

### Example 3. Manufacturing Process for DISU from Choline Hydroxide and Succinic Acid in Reaction Medium Comprising Ethanol or Methanol

Ethanol (or methanol) solution of non-food grade choline hydroxide (around 50 wt. %) was heated to 25-50° C. Crystalline succinic acid is added (molar ratio succinate:choline around 1.01-1.1:2). Optionally, the choline solution, was concentrated by evaporation of the solvent to a residual volume and 2-propanol added to prepare a choline hydroxide solution in 2-propanol, and, then, crystalline succinic acid was added to the prepared choline hydroxide solution. Succinic acid was dissolved in the choline hydroxide solution and the mixture was kept under stirring for 3 hours. Upon completion of the holding time, the solution was filtered through an activated carbon filter cartridge.

The filtered solution was concentrated (by evaporation in the vacuum) to residual volume and transferred to a reactor containing 2-10 vol acetone pre-cooled at 10 to 15° C. The reaction mass was kept under stirring for 3 hours at a temperature of 10 to 15° C.

The formed sediment was filtered through a Nutsche filter, pressed, and rinsed with a 2-propanol:acetone mixture. A moist sediment with residual solvent was obtained. Both the mother-liquors and the rinse solution are collected in a collecting vessel. Crude DISU is formed. 2-propanol was added to the Nutsche filter containing the moist sediment of DISU and the DISU suspension in 2-propanol was transferred to a reactor containing acetone pre-cooled at 10 to 15° C. The reaction mass is kept under stirring for 3 hours, maintaining the temperature within the range 10 to 15° C. The formed sediment was filtered through a Nutsche filter, pressed, and rinsed with a 2-propanol/acetone mixture (1:2). A moist sediment with residual solvent was obtained. Both the mother-liquors and the rinse solution were collected in a collecting vessel. The filtration cake was then dried under vacuum and nitrogen atmosphere. Purified DISU was formed.

After the drying step is complete, the dried DISU sediment on the Nutsche filter is either dissolved in purified water to the target DISU content value (at least 50 wt. %), or formulated as described in Example 4. The analysis of the 50 wt. % DISU solution was analyzed by HPLC and Q-NMR and no o-ethoxylated choline derivatives were detected.

### Example 4. Preparation of Slightly Hygroscopic and Non-Hygroscopic Formulations of DISU

4.1. Water concentrate of DISU

After the final drying step of DISU cake in the Nutsche filter (obtained as described in Examples 1-3) is complete, purified water is added to the dried sediment up to the target DISU content value (72 wt. % DISU). Hygroscopicity of the DISU material was measured following 24 h storage in an incubator at 80% relative humidity and 25° C. The formulation showed an increase in mass around 1.8%. Accordingly, the 72 wt. % DISU concentrate is slightly hygroscopic.

4.2. Solid formulation of DISU

The dry DISU sediment (obtained as described in Examples 1-3) was dissolved in absolute ethanol (2.5-3 wt equivalents of DISU) and the DISU solution was mixed with crystalline succinic acid (0.02-0.03 wt equivalents of DISU), hydroxypropyl cellulose (2.5-3.5 wt equivalents of DISU), microcrystalline cellulose (7-10 wt equivalents of DISU) and silica (0.1-0.2 wt equivalents of DISU). The mixture was airdried and the solid formulation of DISU was obtained.

Hygroscopicity of the solid DISU formulation was evaluated following 24 h storage in an incubator at 80% relative humidity at 25° C. The formulation showed no increase in mass.

1. A method of producing di-choline salt of succinic acid (DISU), comprising
obtaining a solution of choline in water or in a water miscible organic solvent selected from methanol, ethanol, 1- or 2-propanol, wherein the choline solution comprises one or more o-ethoxylated choline derivatives, wherein the amount of said one or more ethoxylated choline derivatives is above 0.3 wt. %;
treating the choline solution with activated carbon (AC), wherein the weight ratio of AC to choline is 100-1:1-100;
adding succinic acid, or succinic acid salt to the choline solution, wherein the molar ratio of choline to succinate is between around 2:1, preferably between around 2:1.01 and around 2:1.1, and allowing succinate to react with choline for at least 10 min at ambient temperature, thereby producing DISU dissolved in water or in the water miscible organic solvent;
wherein the produced solution of DISU contains at least 40 wt. % DISU, preferably 50 wt. % DISU, and 1 at most 0.3 wt. % of the one or more o-ethoxylated choline derivatives.

2. The method of claim 1, wherein the weight ratio of AC to choline is 10-1:1-10.

3. The method according to claim 1 or 2, wherein the choline solution is a solution of choline hydroxide in water or in the water miscible solvent.

4. The method according to claim 1 or 2, wherein the choline solution is a solution of choline salt in water or the water miscible solvent, and wherein the method comprises
treating the choline salt solution with activated carbon (AC), wherein the weight ratio of AC to choline is 100-1:1-100;
adding a base to the choline salt solution, thereby obtaining a solution comprising choline hydroxide and a by-product salt,
adding succinic acid to the solution of choline hydroxide and the salt, wherein the molar ratio of choline to succinate is between 2:1.01 and 2:1.1, and allowing succinate to react with choline for at least 10 min at ambient temperature to form DISU;
removing the by-product salt from the DISU solution.

5. The method of claim 4, wherein the choline salt is selected from choline chloride, choline bicarbonate or choline carbonate.

6. The method according to claim 4 or 5, wherein the base is selected from sodium hydroxide, potassium hydroxide or ammonium hydroxide.

7. The method of any of claims 1 to 6, further comprising a step of obtaining a DISU material with a reduced content of liquid.

8. The method of claim 7, wherein the DISU material with a reduced content of liquid is a DISU solution comprising at least 50 wt. % DISU, preferably around 60-70 wt. % DISU.

9. The method of claim 7, wherein the DISU material with a reduced content of liquid is a DISU precipitate.

10. The method according to claim 9, wherein the DISU precipitate is obtained by
mixing at least 2 volumes of acetone with the DISU solution comprising at least 50 wt. % DISU and obtaining the DISU precipitate by incubating the mixture for 3-6 hours at 10-15° C.;
collecting the DISU precipitate by using Nutsche filtration; and
drying the DISU precipitate collected in the Nutsche filter.

11. The method of claim 10, further comprising dissolving the dried DISU precipitate in water and preparing a liquid formulation of DISU comprising around 70 wt. % DISU.

12. The method of claim 10, further comprising
dissolving the dried DISU precipitate in a water miscible solvent, preferably, methanol, ethanol, 1-propanol or 2-propanol, or a mixture thereof;
mixing the DISU solution with one or more hygroscopicity controlling additives (HCA),
removing the solvent by drying the mixture, and thereby obtaining a non-hygroscopic solid formulation of DISU.

13. The method of claim 12, wherein the one or more HCA are selected from microcrystalline cellulose, ethyl cellulose, hydroxypropyl cellulose, hydroxypropyl methyl cellulose, ethyl methyl cellulose, or a mixture thereof.

14. The method of claim 12 or 13, wherein DISU is mixed with the HCA in the wt./wt. ratio DISU:HCA from 1:3 to 1:10.

15. A liquid formulation comprising at least 40 wt. % DISU, preferably at least 50 wt. % DISU, and at most 0.3 wt. % of one or more o-ethoxylated choline derivatives.

16. The liquid formulation according to claim 15, wherein the formulation comprises from around 70 wt. % DISU.

17. The liquid formulation according to claim 16, wherein the formulation is DISU octahydrate.

18. The liquid formulation according to claim 16, wherein the formulation is DISU heptahydrate.

19. The liquid formulation according to any of claims 15-17, wherein the formulation increases its mass following 24 hours storage at 80% relative humidity and 25° C. by at most 2%.

20. A solid formulation comprising DISU and a hygroscopicity controlling agent (HCA), wherein the wt./wt. ratio of DISU:HCA is from 1:3 to 1:10, and wherein the formulation comprises at most 0.3 wt. % of one or more o-ethoxylated choline derivatives.

21. The solid formulation according to claim 19, wherein the HCA is selected from of microcrystalline cellulose, ethyl cellulose, hydroxypropyl cellulose, hydroxypropyl methyl cellulose, ethyl methyl cellulose, or a mixture thereof.

22. The solid formulation according to claim 19 or 20, wherein said formulation increases its mass following 24 hours storage at 80% relative humidity and 25° C. by at most 0.012%.

23. The formulation according to any of claims 15-21, wherein the DISU material is obtained by the method of any of claims 1 to 14.
